(19) United States
(12) Patent Application Publication
(10) Pub. No.: US 2008/0200963 A1
(43) Pub. Date: Aug. 21, 2008

(54) IMPLANTABLE POWER GENERATOR

(76) Inventors: Benjamin Pless, Atherton, CA (US); Jessica Anne Connor, Palo Alto, CA (US)

Correspondence Address: SCHOX PLC, 730 Florida Street #2, San Francisco, CA, 94110, US

(21) Appl. No.: 12/031,676

(22) Filed: Feb. 14, 2008

Related U.S. Application Data
(60) Provisional application No. 60/901,576, filed on Feb. 15, 2007.

(51) Int. Cl. A61N 1/02

(52) U.S. Cl. 607/36

(57) ABSTRACT

The implantable power generator of the preferred embodiments includes a cardiac restraint device and a generator that generates electrical energy in response to a mechanical force. The generator is coupled to the cardiac restraint device such that it receives a mechanical force and generates electrical energy in response to the mechanical force. The generator of the preferred embodiments includes a transducer that generates electrical energy in response to a mechanical force and an electrode coupled to the transducer that collects the electrical energy generated by the transducer. The implantable power generator is preferably designed for the power generation field, and more specifically to a new and useful implantable power generator coupled to a cardiac restraint device. The implantable power generator, however, may be alternatively used in any suitable environment and for any suitable reason.

### CROSS-REFERENCE TO RELATED APPLICATIONS

This application claims the benefit of U.S. Provisional Application No. 60/901,576 filed on 15 Feb. 2007 and entitled “Implanted Power Generator”, which is incorporated in its entirety by this reference.

### TECHNICAL FIELD

This invention relates generally to the power generation field, and more specifically to a new and useful implantable power generator coupled to a cardiac restraint device in the power generation field.

### BRIEF DESCRIPTION OF THE FIGURES

FIG. 1 is the implantable power generator of a preferred embodiment of the invention;

FIG. 2 is a cross-sectional view taken along the line A-A′ in FIG. 3 of the generator of a preferred embodiment of the invention;

FIG. 3 is a top view of the generator of a preferred embodiment of the invention;

FIG. 4 is a cross-sectional view taken along the line B-B′ in FIG. 3 of the generator of a preferred embodiment of the invention;

FIG. 5 is a block diagram of the circuit of a preferred embodiment of the invention;

FIG. 6 is the implantable power generator of a preferred embodiment of the invention with the third variation of the cardiac restraint device; and

FIG. 7 is the implantable power generator of a preferred embodiment of the invention with the fourth variation of the cardiac restraint device.

### DESCRIPTION OF THE PREFERRED EMBODIMENTS

The following description of preferred embodiments of the invention is not intended to limit the invention to these preferred embodiments, but rather to enable any person skilled in the art to make and use this invention.

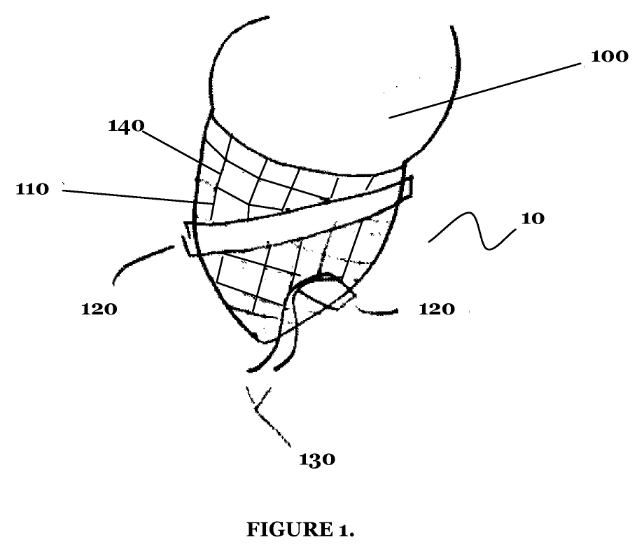

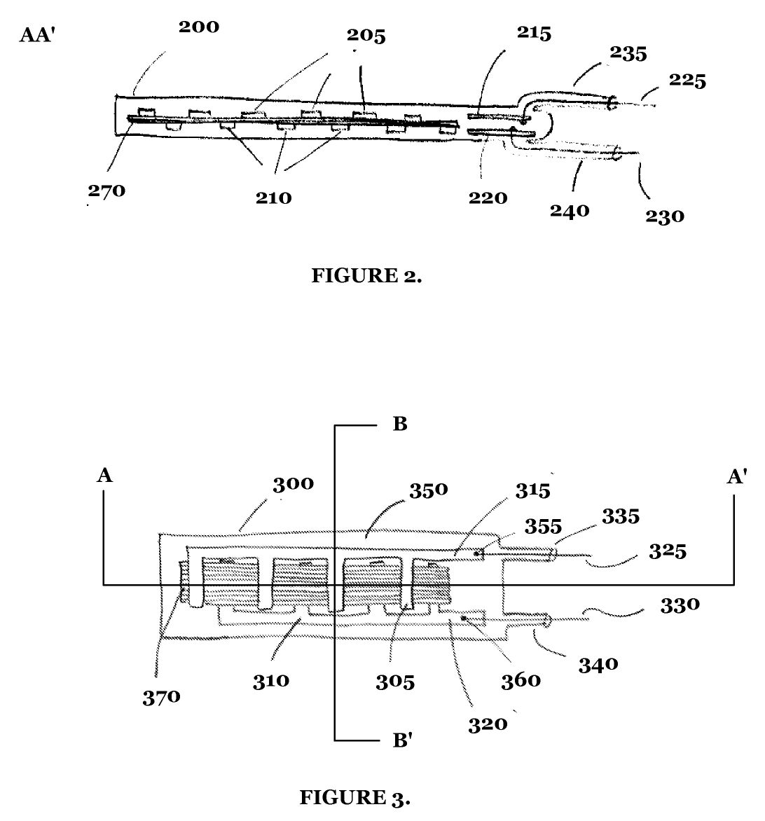

As shown in FIG. 1, the implantable power generator 10 of the preferred embodiments includes a cardiac restraint device 110 and a generator 120 that generates electrical energy in response to a mechanical force. The generator 120 is coupled to the cardiac restraint device 110 such that it receives a mechanical force, preferably harvested from a heart, and generates electrical energy in response to the mechanical force, preferably for powering medical devices. As shown in FIG. 2, the generator 200 of the preferred embodiments includes a transducer 270 that generates electrical energy in response to a mechanical force and electrodes 205 and 210 coupled to the transducer 270 that collects the electrical energy generated by the transducer 270. The implantable power generator 10 is preferably designed for the power generation field, and more specifically to a new and useful implantable power generator coupled to a cardiac restraint device 110. The implantable power generator 10, however, may be alternatively used in any suitable environment and for any suitable reason.

The cardiac restraint device 110 of the preferred embodiments functions to supports a patient's heart 100 and, more specifically, to reduce or limit ventricular dilation of a heart 100 and thereby preferably slow or stop the progression of dilated cardiomyopathy (DCM) and heart failure. A cardiac restraint device is a therapeutic device used to treat heart failure, or incipient heart failure. It is typically formed from netting or other material that is put around the heart to provide mechanical support as the heart beats and to reduce the volume of blood in the ventricles.

The cardiac restraint device 110 is preferably made from a biocompatible material such as silicon polymers, Polytetrafluoroethylene (PTFE), Marlex fabric, PET-polyester, or nitinol or other metals. The cardiac restraint device may be comparatively non-resilient (as disclosed for example in U.S. Pat. No. 5,702,303, which is hereby incorporated in its entirety by this reference) or resilient (see for example U.S. Pat. No. 6,595,912, which is hereby incorporated in its entirety by this reference). The cardiac restraint device 110 is preferably flexible such that it can conform to the shape of the heart 100 as the heart 100 beats and while allowing the heart 100 to perform normal functions. The cardiac restraint device 110 is preferably loose enough to permit proper cardiac function, while tight enough to limit ventricular expansion.

The cardiac restraint device 110 is preferably a mesh. The mesh preferably includes a plurality of lattice members 140 that provide the basic structure of cardiac restraint device 110. The lattice members 140 are preferably compliant members, but may alternatively be woven fibers or any other suitable material. The lattice members 140 may have any suitable geometry and may have any suitable material properties such that the cardiac restraint device 110 is flexible to allow normal function of the heart 100 while sufficiently restricting dilatation of the heart 100. Each individual lattice member 140 may have a distinct geometry and material properties or alternatively, groups of lattice members 140 may have the same geometry and material properties. For example, more flexible lattice members 140 may be coupled to less flexible or rigid lattice members 140. The cardiac restraint device 110 may, however, be a solid material such as a film or fabric. The solid material is preferably compliant and may include reinforcing members or elements of higher rigidity to provide support to the cardiac restraint device 100 and/or limit the expansion of the device 110 to prevent dilatation of the heart 100. The cardiac restraint device 110 in this variation may further include multiple layers of solid material. Each layer may have a distinct geometry and material properties.

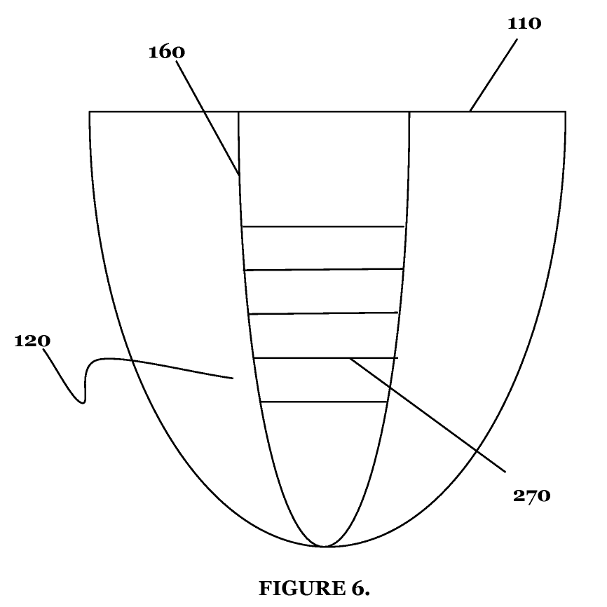

As shown in FIG. 6, in a first variation, the cardiac restraint device 110 is adjustable. In this variation, the geometry and/or the material properties of the cardiac restraint device 110 are adjustable. The cardiac restraint device 110 of this variation preferably includes an adjustment element 160. The properties of the cardiac restraint device 110 and/or the adjustment element 160 may be adjustable before or after implantation. The cardiac restraint device 110 is preferably adjusted after implantation in order to position and fit the cardiac restraint device properly to the heart 100. In a first version of the first variation, the geometry or fit of the cardiac restraint device 110 and/or the adjustment element 160 is adjusted manually. In a second version of the first variation, the geometry or fit of the cardiac restraint device 110 and/or the adjustment element 160 is adjusted automatically. In the second version, the cardiac restraint device 110 and/or the adjustment element 160 is preferably made from a shape memory material, a thermally activated material, or any other suitable material or actuator such as piezoelectric fibers, piezo-active polymers (such as PVDF), electro active polymers, nano-generators, or MEMS based (or simply miniaturized) coils and magnet style generators. In this variation, the electrical energy generated by the generator 120 may be used to supply energy to the adjustment element 160.

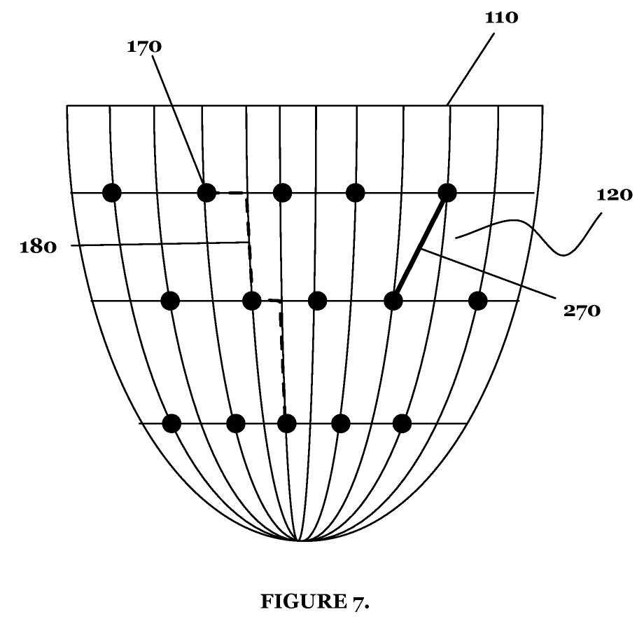

In a second variation, as shown in FIG. 7, the cardiac restraint device 110 preferably includes a plurality of therapeutic electrodes 170 that function to record, stimulate, sense or monitor cardiac activity, perform any other suitable function, or any combination thereof. The plurality of therapeutic electrodes 170 may each be independently designed to record, stimulate, sense or monitor cardiac activity, perform any other suitable function, or any combination thereof or alternatively, two or more therapeutic electrodes 170 may be grouped and used to perform the same function. The plurality of therapeutic electrodes 170 preferably contact the heart and in conjunction with circuitry, for example pacemaker circuitry (not shown), stimulate the heart in a therapeutic fashion to regulate the beating of the heart to maintain or return the heart to an adequate heart rate, a resynchronized heart rhythm, and/or a regular heartbeat pattern. In this variation, the cardiac restraint device 110 preferably includes interconnects 180 that couple to the plurality of therapeutic electrodes 170 and provide electrical energy. The interconnects 180 are preferably incorporated in the mesh or solid material of the cardiac restraint device 110. In this variation, the electrical energy generated by the generator 120 may be used to supply energy to the plurality of therapeutic electrodes 170.

Although the cardiac restraint device 110 preferably includes one or both of these variations, the cardiac restraint device may be any suitable device to reduce or limit ventricular dilation of a heart 100 and thereby preferably slow or stop the progression of dilated cardiomyopathy (DCM) and heart failure.

As shown in FIG. 1, the generator 120 of the preferred embodiment is coupled to the cardiac restraint device 110 and functions to generate electrical energy in response to a mechanical force, preferably of the heart. The mechanical force is preferably generated by the beating or movement of the heart 100 and is preferably delivered directly to the generator 120 or to the generator 120 via the cardiac restraint device 110. The generator is preferably coupled to the cardiac restraint device in one of several variations. In a first variation, as shown in FIG. 1, the electrical energy generator 120 wraps around the heart in a spiral formation. Other configurations including longitudinal or latitudinal strips are also possible as long as the electrical energy generator is positioned such that it flexes when the heart beats. In a second variation, the generator 120 is incorporated in the cardiac restraint device 110. In this variation, the generator 120 may be woven, molded, coupled between layers of materials, or incorporated into the cardiac restraint device in any other suitable fashion. The generator 120 may be included in a lattice member 140 or may make up one or more of the lattice members 140 and couple to other lattice members 140.

In a third variation, as shown in FIG. 6, the generator 120 may be incorporated into the adjustment element 160 of the cardiac restraint device 110. In the case where the adjustment element 160 is preferably made from a shape memory material, a thermally activated material, or any other suitable material or actuator such as piezoelectric fibers, piezo-active polymers (such as PVDF), electro active polymers, nano-generators, or MEMS based (or simply miniaturized) coils and magnet style generators, the generator 120 may utilize the same or similar materials to generate an electrical energy in response to a mechanical force. In this variation, the adjustment element 160 may further function to generate an electrical energy in response to a mechanical force.

In a fourth variation, the generator 120 includes a coupling element that couples the transducer to a cardiac restraint device such that the transducer is coupled to coupling element such that it receives a mechanical force and generates electrical energy in response to the mechanical force. Any of the above variations described may include any suitable coupling element to couple the transducer and/or generator 120 to the cardiac restraint device. Although the generator 120 is preferably coupled to the cardiac restraint device in one of these variations, the generator 120 may be coupled to the cardiac restraint device 110 in any suitable fashion such that it receives a mechanical force and generates electrical energy in response to the mechanical force.

As shown in FIG. 2, the generator 200 of the preferred embodiments includes a transducer 270 that generates electrical energy in response to a mechanical force and electrode 205 and 210 coupled to the transducer 270 that collects the electrical energy generated by the transducer 270. The generator is preferably one of several variations.

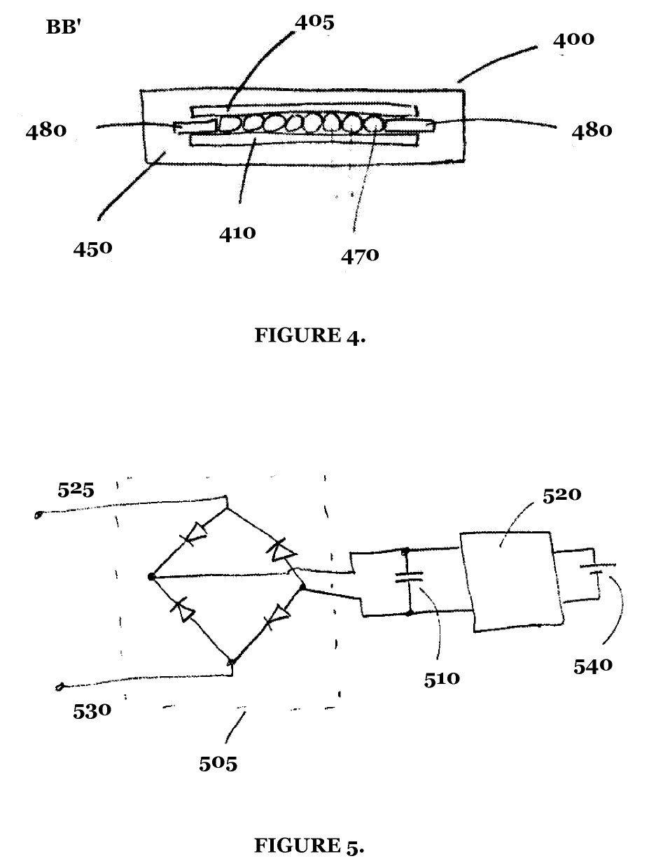

In a first variation of the generator, as shown in FIG. 2, the transducer 270 that converts the mechanical motion to electricity is preferably one or more piezoelectric fibers, which generate an electric potential in response to mechanical stress or force. The mechanical force is preferably generated by the beating or movement of the heart 100 and is preferably delivered directly to the transducer 270, to the transducer 270 via the generator 120, or to the transducer 270 via the generator 120 via the cardiac restraint device 110. Other electromechanical generators may be employed including piezo-active polymers (such as PVDF), nano-generators, or MEMS based (or simply miniaturized) coils and magnet style generators. Such transducers may include piezoelectrical materials, electro-active polymers, electromagnetic elements, nano-generators, or other transduction means. In this variation, the transducer 270 preferably runs almost the entire length of the generator 200, but may alternatively have any suitable geometry and run any suitable length or width of the generator 200. The generator 200 preferably includes two electrodes: top electrodes 205 and bottom electrodes 210, which make contact with opposite sides of the transducer 270. When the heart motion causes the transducer 270 to flex, a voltage appears across the transducer 270 and is collected by the electrodes 205 and 210. As shown in FIG. 3, the piezoelectric fibers of the transducer 370 preferably lay side-by-side with one electrode 305 shown on top of the transducer 370, and the other electrode 310 shown beneath the transducer 370. Although comparatively few extensions of the electrodes 305, 310 over the transducer 370 are shown for clarity, any suitable numbers and configurations of the extensions of the electrodes 305, 310 over the transducer 370 may be utilized. As shown in FIG. 4, the piezoelectric fibers of the transducer 470 are preferably sandwiched between the electrodes 405 and 410. An insulating separator 480 helps keep the electrodes from shorting to each other. The insulating separator 480 is preferably made of a polymer such as polyimide.

In a second variation, as shown in FIG. 6, the generator 120 preferably includes a transducer 270 that generates electrical energy in response to a mechanical force and an electrode coupled to the transducer 270 that collects the electrical energy generated by the transducer 270. The transducer 270 that converts the mechanical motion to electricity is preferably a portion of the adjustment element 160. The adjustment element 160 is preferably made from a shape memory material, a thermally activated material, or any other suitable material or actuator such as piezoelectric fibers, piezo-active polymers (such as PVDF), electro-active polymers, nano-generators, or MEMS based (or simply miniaturized) coils and magnet style generators. In this variation, at least one of the elements that change the shape or the properties of the cardiac restraint device 110 and/or adjustment element 160, are then used to generate electrical energy in response to a mechanical force. In this variation, the electrical energy generated by the generator 200 may be used to supply energy to the adjustment element 160 to change shape, alter material properties, etc.

As shown in FIG. 1, the generator 120 of the preferred embodiment may further include insulated wires 130 that function to conduct the electrical energy generated to an implanted electronic device or any other suitable device requiring power (not shown). Additionally, the electrodes 205 and 210 (labeled 305 and 310 in FIG. 3), in the first variation of the generator 200, as shown in FIG. 2, may terminate in tabs 215 and 220 (labeled 315 and 320 in FIG. 3) respectively, which are preferably coupled to wires 225 and 230 (labeled 325 and 330 in FIG. 3 and 525 and 530 in FIG. 5) respectively, which carry the generated electricity to a circuit (as shown in FIG. 5) that functions to condition the voltage generated by the transducer 270 to be used in an implanted medical device or any other suitable device that requires power. The wires 225 and 230 are preferably coiled conductors in an insulator 235, 240 (labeled 335 and 340 in FIG. 3) made of silicone rubber or polyurethane suitable for long term implantation.

The generator 200 of the preferred embodiment may further include a sheath (labeled 350 in FIG. 3 and 450 in FIG. 4) that functions to protect the generator 200 from the stresses of the implant environment. As shown in FIG. 2, the transducer 270, electrodes 205, 210 and tabs 215, 220 or any combination thereof are preferably encased in a flexible, biocompatible polymer sheath 350 preferably made of polyimide, polyurethane or silicone rubber or a combination thereof. The sheath 350 may be assembled and glued or insert molded.

As shown in FIG. 5, the generator of the preferred embodiment further includes a circuit, coupled to the electrode, that converts the electrical energy collected by the electrode (205, 210, 305, and/or 310) into a substantially DC current. The wires 525, 530 that carry the electrical energy generated by the piezoelectric fibers 270 preferably connect with an electrical circuit. As shown in FIG. 5, a first variation of the circuit uses the generated voltage to charge a battery 540. The battery 540 may be used to power a pacemaker, defibrillator, neurostimulator, pump, or other implanted device. The inputs to the circuit are wires 525 and 530 (which are continuous with wires 225 and 230 respectively of FIG. 2) that carry the generated electricity. The voltage waveform (as measured between the wires 525 and 530) is a low frequency oscillating signal that varies with the heart rate of the patient. In the illustrative embodiment, the oscillatory voltage is preferably converted to a quasi-DC (direct current) voltage through the action of four diodes configured as a bridge 505 and an input capacitor 510 to store the DC voltage. The resultant DC voltage can range from below the battery 540 voltage to above the battery 540 voltage and generally needs to be conditioned by a voltage conditioning circuit 520 before it can be used to charge the battery. A “buck-boost” circuit employing an inductor or a comparable switched capacitor scheme (see for example U.S. Pat. No. 6,198,645, which is incorporated in its entirety by this reference) is preferred as a means to efficiently convert the variable voltage on the input capacitor 510 to a voltage suitable for charging a battery 540. Battery 540 is preferably a rechargeable battery 540, but any other electrical energy storage devices may be used such as a capacitor. The power available from the circuit can be used to monitor the mechanical condition of the heart. In particular, the voltage on capacitor 510 can be monitored while under constant load from circuit 520 to determine the relative amount of mechanical energy generated by the heart. Such information may be used to monitor the health and condition of the patient's heart.

As a person skilled in the art will recognize from the previous detailed description and from the figures and claims, modifications and changes can be made to the preferred embodiments of the invention without departing from the scope of this invention defined in the following claims.

We claim:

1. An implantable power generator comprising:
cardiac restraint device adapted to restrain a heart; and
a generator coupled to the cardiac restraint device and adapted to generate electrical energy in response to a mechanical force from the heart.

2. The implantable power generator of claim 1 wherein the cardiac restraint device is coupled to the heart such that the generator is coupled to a first portion of the heart and coupled to a second portion of the heart that moves relative to the first portion thereby creating the mechanical force.

3. The implantable power generator of claim 2 wherein the cardiac restraint device is a mesh that includes a plurality of lattice members that provide the basic structure of cardiac restraint device; wherein the generator is coupled to at least one lattice member.

4. The implantable power generator of claim 2 wherein the cardiac restraint is a solid material and the generator is incorporated in the solid material.

5. The implantable power generator of claim 1 further comprising a battery coupled to the generator.

6. The implantable power generator of claim 1 further comprising a medical device powered by the generator.

7. The implantable power generator of claim 6 wherein the medical device is a cardiac rhythm management device.

8. The implantable power generator of claim 6 wherein the medical device is a nerve stimulation device.

9. The implantable power generator of claim 6 wherein the medical device is a drug pump.

10. The implantable power generator of claim 1 wherein the cardiac restraint device includes an adjustment element adapted to adjust the physical relationship between the cardiac restraint device and the heart.

11. The implantable power generator of claim 10 wherein the adjustment element is powered by the generator.

12. The implantable power generator of claim 1 wherein the cardiac restraint device includes a therapeutic electrode adapted to stimulate at least a portion of the heart.

13. The implantable power generator of claim 12 wherein the therapeutic electrode is powered by the generator.

14. The implantable power generator of claim 1 wherein the generator includes:
a transducer adapted to generate electrical energy in response to a mechanical force; and
an electrode coupled to the transducer and adapted to collect the electrical energy generated by the transducer.

15. The implantable power generator of claim 14 wherein the transducer is a piezoelectric fiber.

16. The implantable power generator of claim 14 wherein the transducer is an electro-active polymer.

17. The implantable power generator of claim 14 wherein the transducer is a microelectromechanical system.

18. The implantable power generator of claim 14 wherein the generator further includes a circuit, coupled to the electrode, that converts the electrical energy collected by the electrode into a substantially DC voltage.

19. The implantable power generator of claim 18 wherein the circuit includes a capacitor that stores the substantially DC voltage.

20. The implantable power generator of claim 18 wherein the circuit is coupled to a device and the substantially DC voltage is used as an indicator of the health of the heart.

21. An implantable power generator that can be coupled to a cardiac restraint device, the implantable power generator comprising:
a transducer that generates electrical energy in response to a mechanical force;
an electrode coupled to the transducer that collects the electrical energy generated by the transducer; and
a coupling element that couples the transducer to a cardiac restraint device such that the transducer receives a mechanical force from a heart and generates electrical energy in response to the mechanical force.
